(19) United States
(12) Patent Application Publication
(10) Pub. No.: US 2010/0311968 A1
(43) Pub. Date: Dec. 9, 2010

(54) DEPROTECTION OF BOC-PROTECTED COMPOUNDS

(76) Inventors: Jason Chi-Chung Choy, Alameda, CA (US); Saul Jaime-Figueroa, Morris Plains, NJ (US)

Correspondence Address: Grant D. Green, Patent Law Department;Roche Palo Alto LLC, M/S A2-250,, 3431 Hillview Avenue, Palo Alto, CA, 94304, US

(21) Appl. No.: 12/797,343

(22) Filed: Jun. 9, 2010

Related U.S. Application Data
(60) Provisional application No. 61/185,431, filed on Jun. 9, 2009.

(51) Int. Cl. C07F 7/18; C07C 45/65; C07C 37/055; C07F 5/02; C07C 27/00; C07C 51/09; C07D 213/55; C07D 235/26

(52) U.S. Cl. 544/229; 568/433; 568/716; 568/6; 568/812; 562/493; 546/327; 548/306.4

(57) ABSTRACT

Organic compounds having t-butyl ester or BOC carbonate protecting groups are effectively deprotected by heating in a fluorinated alcohol solution.

### CROSS REFERENCE TO RELATED APPLICATIONS

This application is entitled to the benefit of U.S. provisional patent application Ser. No. 61/185,431 filed on Jun. 9, 2009, the disclosure of which is incorporated herein by reference.

### FIELD OF THE INVENTION

This invention relates generally to the field of synthetic chemistry. More particularly, the invention relates to methods for deprotecting protected organic compounds that have a t-butyl ester or a BOC carbonate, using fluorinated alcohols.

### BACKGROUND OF THE INVENTION

Among various protecting groups for carboxylic acids, the tert-butyl (t-Bu) group is perhaps the most widely used due to its stability towards a variety of reagents and reaction conditions (T. W. Greene, P. G. M. Wuts, “Protective Groups in Organic Synthesis” (3^{rd} ed; John Wiley and Sons, New York 1999); P. Kocienski, “Protecting Groups” (3^{rd} ed; Thieme Verlag, Stuttgart, 2000)). As a result, cleavage of the t-butyl group remains of prime importance in organic synthesis. Deprotection of t-butyl esters is generally achieved by employing strong protic acids such as trifluoroacetic and hydrochloric acid (J. G. Gleason et al., J Am Chem Soc (1977) 99:2353), or Lewis acid catalyzed conditions using ZnBr₂ (Y.-q. Wu et al., Tetrahedron Lett (2000) 41:2847-49), CeCl₃ (G. Bartoli et al., J Org Chem (2001) 66:4430), and SiO₂ (R. W. Jackson, Tetrahedron Lett (2001) 42:5163). In the less stable case of t-Bu-carbonates, basic conditions (S. El-Kazzouli et al., Tetrahedron Lett (2006) 47:8575; S. P. Govek et al., J Am Chem Soc (2001) 123:9468) using Na₂CO₃ have also been described in the literature for this deprotection. Similarly, the use of a Me₃SiOTf-lutidine mixture has been employed (A. B. Jones et al., J Org Chem (1990) 55:2786; M. Duan et al., Angew Chem Int Ed (2001) 40:3632) under very mild conditions for t-butyl ester and carbonate deprotection. Methods involving the thermolytic neat-cleavage (>200° C.) of tert-butyl esters have also been reported (L. H. Klemm et al., J Org Chem (1962) 27:519), but these are very harsh condition for many substrates. Because each of these methods require the fastidious use of specific reagents and/or suffer of drawbacks due to substrate sensitivity to acids, attempts to find alternative practical conditions are still very desirable.

### SUMMARY OF THE INVENTION

We have now invented a method for removing t-butyl ester and BOC carbonate protecting groups from organic compounds using fluorinated alcohols. The reaction conditions are neutral and do not require additional reagents (apart from solvents). Thus, the product is recovered by a simple solvent evaporation without any work up and in some cases, no further purification is needed.

One aspect of the invention is a method for deprotecting a protected compound having a t-butyl ester or BOC carbonate protecting group, by dissolving a protected compound having a t-butyl ester or BOC carbonate protecting group in a fluorinated alcohol to form a solution; and heating the solution for a period of time sufficient to remove BOC or t-butyl from said protected compound, thereby providing a deprotected compound.

### DETAILED DESCRIPTION OF THE INVENTION

All publications cited in this disclosure are incorporated herein by reference in their entirety.

### Definitions

Unless otherwise stated, the following terms used in this Application, including the specification and claims, have the definitions given below. It must be noted that, as used in the specification and the appended claims, the singular forms “a”, “an,” and “the” include plural referents unless the context clearly dictates otherwise.

The term “t-butyl” refers to the radical (CH₃)₃C—, while “t-butyl ester” refers to a compound having the group (CH₃)₃C—OC(O)—. The term “BOC” refers to the radical t-butoxy-carbonyl, (CH₃)₃COC(O)—. The term “BOC carbonate” refers to the radical (CH3)3COC(O)O—.

The terms “protected compound” refers to an organic compound that comprises a t-butyl ester and/or a BOC carbonate. It is possible for a protected compound to have both a t-butyl ester and a BOC carbonate simultaneously.

The term “deprotected compound” refers to a compound from which BOC and/or t-butyl has been removed. Note that a deprotected compound within the scope of this invention may still retain other protecting groups, which are generally undisturbed by the method of the invention.

The term “fluorinated alcohol” refers to compounds of the formula R¹R²R³C—OH, where R¹ is a fluorinated lower alkyl radical, and R² and R³ are each independently H or a fluorinated lower alkyl radical. Exemplary fluorinated alcohols include, without limitation, 2,2,2-trifluoro-ethanol (“TFE”), 1,1,1,3,3,3-hexafluoroisopropanol (“HFIP”), 3,3,4,4,4-pentafluorobutan-2-ol (“PFB”), and the like.

The term “lower alkyl” refers to monovalent hydrocarbon radicals composed of carbon and hydrogen, and having no unsaturation. Lower alkyl radicals may be straight or branched, and contain from 1 to 6 carbon atoms, inclusive.

The term “fluorinated lower alkyl” refers to a lower alkyl radical in which one or more hydrogen atoms has been replaced by fluorine. Exemplary fluorinated lower alkyl radicals include, without limitation, CF₃—, CHF₂—, CF₃CF₂—, CHF₂CF₂—, and the like.

The term “labile” as used herein refers to the relative bond strength and ease of removing the BOC and/or t-butyl protecting group.

All patents and publications identified herein are incorporated herein by reference in their entirety.

### General Method

The invention provides a new, practical method to cleanly deprotect oxygen atoms in organic compounds protected with BOC or t-butyl by using a fluorinated alcohol such as 2,2,2-trifluoroethanol (TFE) or hexafluoroisopropanol (HFIP) as a solvent, in quantitative yields.

In practice, a protected compound is first dissolved in a fluorinated alcohol such as TFE or HFIP. The quantity of fluorinated alcohol required to dissolve the protected compound will depend in general on the solubility of the compound. As a starting point, one may begin with a ratio of about 1 mmol protected compound to about 5 mL of fluorinated alcohol, and adjust the ratio by routine experimentation to maximize results. If the protected compound is not sufficiently soluble in a fluorinated alcohol, a co-solvent such as benzene, toluene, pyridine, dimethylsulfoxide, N-methylpyrrolidine, dichloromethane, chloroform, dioxane, tetrahydrofuran, or the like may be added.

The solution may be heated by convention methods, for example by gas burner, oil bath, and the like. Preferably, the solution is heated using a microwave radiator, such as a Biotage INITIATOR™ 60 focused microwave reactor. The solution is preferably stirred during heating.

In general, the reaction times and temperatures necessary will depend upon the nature of the compound to be deprotected and the heating method. When using TFE or HFIP with most protected compounds and conventional heating at the reflux temperature of the solvent, a reaction time of about 30 minutes to about 48 hours is generally necessary. When using TFE or HFIP with most protected compounds and microwave heating, a temperature of between about 80° C. and about 200° C. is sufficient, preferably between about 100° C. and 170° C. Reaction times may range, in general, from about 1 minute to about 6 hours, typically from about 1 hour to about 4 hours. Optimal reaction times and selection of fluorinated alcohol are determined by routine experimentation, for example following the Examples set forth below. In general, BOC and t-butyl groups that are less labile can be removed by (a) increasing the reaction time, (b) switching to a more reactive fluorinated alcohol (for example, from TFE to HFIP), and/or (c) increasing the temperature.

After completion of the deprotection reaction, the fluorinated alcohol may be removed by evaporation, and the deprotected compound recovered and purified by convention methods, for example, by column chromatography, HPLC, recrystallization, and the like. The fluorinated alcohol is preferably recovered and reused.

### EXAMPLE 1

### Deprotection of O-BOC Compounds

(A) A solution of O-(t-butyl)-O′-(4-formylphenyl)-carbonate (1 mmol) in TFE (5 mL) was placed in a sealed microwave vial. The reaction mixture was heated at 100° C. in a Biotage-Initiator™ Sixty microwave reactor until the disappearance of starting material was complete (about 30 min). After cooling to RT, the mixture was evaporated to dryness under reduced pressure to provide 4-hydroxybenzaldehyde in 98% yield. Mp=115-117° C. (lit. 113-117° C.); ¹H nmr (400 MHz, CDCl₃) δ ppm: 6.35 (br. s, 1H), 6.99 (d, J=8.59 Hz, 2H), 7.83 (d, J=8.59 Hz, 2H), 9.87 (s, 1H); ¹³C nmr (400 MHz, CDCl₃) δ ppm: 191.24, 161.59, 132.53, 129.90, 116.03; MS ESI: m/z (%) 123 (M+H⁺, 100); Anal. calc. for C₇H₆O₂: C-68.85; H-4.95; found: C-68.84, H-4.87.

(B) Similarly, proceeding as in part (A) above but substituting O-(t-butyl)-O′-(2,6-di-methylphenyl)-carbonate for O-(t-butyl)-O′-(4-formylphenyl)-carbonate, and heating for 1 h, the compound 2,6-dimethylphenol was produced in 96% yield. Mp 43-45° C. (lit. 44-45° C.); ¹H NMR (300 MHz, CDCl₃) δ ppm 2.27 (s, 6 H), 4.63 (s, 1 H), 6.68-6.84 (m, 1 H), 7.00(d, J=7.54 Hz, 2 H); ¹³C NMR (300 MHz, CDCl₃) δ ppm 152.11, 128.56, 122.91, 120.17, 15.82; MS EI: m/z (%) 122 (M⁺, 100).

(C) Proceeding as in part (A) above, but substituting O-(t-butyl)-O′-[4-(4,4,5,5-tetra-methyl-[1,3,2]dioxaborolan-2-yl)-phenyl]-carbonate for the starting material and heating for 1 h, the compound 4-(4,4,5,5-tetramethyl-[1,3,2]dioxaborolan-2-yl)-phenol was produced in 96% yield. Mp 115-117° C. (lit. 106-107° C.); ¹H NMR (400 MHz, CDCl₃) δ ppm 1.35 (s, 12 H), 5.40 (br. s., 1 H), 6.83 (d, J=8.59 Hz, 2 H), 7.72 (d, J=8.59 Hz, 2 H); ¹³C NMR (400 MHz, CDCl₃) δ ppm 136.78, 132-120 (br. m) 114.82, 83.69, 77.00, 24.80; MS EI: m/z (%) 220 (M⁺, 98); Anal. calc. for C₁₂H₁₇BO₃: C-65.49; H-7.79. Found: C-65.48; H-7.81.

(D) Proceeding as in part (A) above, but substituting O-(t-butyl)-O′-[2-(3-trifluoro-methylphenyl)-ethyl]-carbonate for the starting material, and heating for 1 h, the product 2-(3-trifluoromethyl-phenyl)-ethanol was produced in 95% yield (estimated by nmr due to low boiling point). Oil; ¹H NMR (400 MHz, CDCl₃) δ ppm 1.74 (t, J=5.56 Hz, 1 H), 2.91 (t, J=6.57 Hz, 2 H), 3.83-3.91 (m, 2 H), 7.36-7.55 (m, 4 H); ¹³C NMR (400 MHz, CDCl₃) δ ppm 139.61, 132.41, 130.78 (q, J=32.2 Hz), 128.88, 124.13 (q, J=272.25 Hz) 25.63, 123.29, 63.18, 38.81; MS ESI: m/z (%) 189 (M⁺−H, 100).

(E) Proceeding as in part (A) above, but substituting O-(t-butyl)-O′-[5-(t-butyl-dimethyl-silanyloxymethyl)-2-(2,4-dioxo-3,4-dihydro-2H-pyrimidin-1-yl)-4-hydroxy-tetrahydrofuran-3-yl]-carbonate for the starting material, and heating for 1 h, the product 1-[5-(t-butyl-dimethyl-silanyloxymethyl)-3,4-dihydroxy-tetrahydrofuran-2-yl]-1H-pyrimidine-2,4-dione was obtained in 97% yield. Mp, sample shrinks at 100° C. (lit. 105-108° C.); ¹H NMR (400 MHz, CDCl₃) δ ppm 0.12 (s, 6 H), 0.93 (s, 9 H), 3.86 (d, J=10.61 Hz, 1 H), 4.04 (d, J=13.64 Hz, 1 H), 4.12-4.34 (m, 3 H), 5.67 (d, J=8.59 Hz, 1 H), 5.91 (d, J=2.02 Hz, 1 H), 8.10 (d, J=8.59 Hz, 4 H); ¹³C NMR (400 MHz, CDCl₃) δ ppm 163.97, 151.25, 140.47, 102.04, 90.44, 84.96, 75.66, 69.19, 61.73, 25.89, −5.57; MS ESI: m/z (%) 357 (M−H⁻, 100).

### EXAMPLE 2

### Deprotection of t-Butyl-Esters

(A) A solution of 5-bromo-2-chloro-benzoic acid t-butyl ester (1 mmol) in HFIP (5 mL) was placed in a sealed microwave vial. The reaction mixture was heated to 100° C. in a Biotage-Initiator™ Sixty microwave reactor until the disappearance of starting material was complete (about 2 h). After cooling to RT, the mixture was evaporated to dryness under reduced pressure to provide 4-bromo-2-chloro-benzoic acid in 96% yield. Mp 170-172° C.; ¹H NMR (300 MHz, DMSO-d₆) δ ppm 7.65 (dd, J=8.69, 1.89 Hz, 1 H), 7.74 (d, J=8.31 Hz, 1 H), 7.85 (d, J=1.89 Hz, 1 H), 13.59 (br. s., 1 H); ¹³C NMR (400 MHz, DMSO-d₆) δ ppm 165.93, 132.99, 132.86, 132.41, 130.55, 130.39, 125.02; MS ESI: m/z (%) 233 (M⁺−H, 100); Anal. calc. for C₇H₄BrClO₂: C-35.71; H-1.71. Found: C-35.83; H-1.42.

(B) Similarly, proceeding as in part (A) above but substituting 5-bromopyridine-2-carboxylic acid t-butyl ester for the starting material, and heating for 3 h, the product 5-bromopyridine-2-carboxylic acid was obtained in 95% yield. Mp 175-177° C. (lit. 173-174° C.); ¹H NMR (400 MHz, DMSO-d₆) δ ppm 7.97 (d, J=8.08 Hz, 1 H), 8.23 (dd, J=8.59, 2.53 Hz, 1 H), 8.83 (d, J=2.53 Hz, 1 H), 13.41 (br. s., 1 H); ¹³C NMR (400 MHz, DMSO-d₆) δ ppm 165.52, 150.33, 147.12, 140.05, 126.33, 124.10; MS EI: m/z (%) 201 (M⁺, 25); Anal. calc. for C₆H₄BrNO₂: C-35.67; H-2.00; N-6.93. Found: C-35.86; H-1.85, N-6.72.

(C) To a solution of 1-benzyl-1,3-dihydrobenzoimidazol-2-one (224 mg, 1 mmol) in DMF (3 mL) was added NaH (44 mg, 1.1 mmol, 60% dispersion in oil) at 0° C., and the mixture stirred for 1 h. To this was added t-butyl bromoacetate (214 mg, 1.1 mmol), and the reaction mixture stirred at RT for 12 h. The reaction mixture was then poured into water (50 mL), extracted with EtOAc (2×50 mL), the organic layers combined, dried over Na₂SO₄, and the solvent removed under vacuum. The crude product was purified by column chromatography (SiO₂, hexanes-EtOAc 9:1) to provide (3-benzyl-2-oxo-2,3-dihydrobenzoimidazol-1-yl)-acetic acid t-butyl ester (313 mg, 97% yield). Mp=99-100° C.; ¹H NMR (400 MHz, CDCl₃) δ ppm 1.47 (s, 9 H), 4.58 (s, 2 H), 5.11 (s, 2 H), 6.88 (t, J=6.82 Hz, 2 H), 6.95-7.10 (m, 2 H), 7.19-7.36 (m, 5 H); ¹³C NMR (CDCl₃) δ ppm 166.53, 154.08, 135.89, 129.03, 128.97, 128.47, 127.38, 127.08, 121.40, 121.24, 108.24, 107.39, 82.43, 44.66, 42.88, 27.73; MS ESI m/z (%) 339 (M+H⁺, 55); Anal. calc. for C₂₀H₂₂N₂O₃: C-70.99; H-6.55; N-8,28. Found: C-70.63; H-6.39; N-8.24.

A solution of (3-benzyl-2-oxo-2,3-dihydrobenzoimidazol-1-yl)-acetic acid t-butyl ester (313 mg) in TFE (5 mL) was placed in a sealed microwave vial. The reaction mixture was heated to 150° C. in a Biotage-Initiator™ Sixty microwave reactor until the disappearance of starting material was complete (about 1 h). After cooling to RT, the mixture was evaporated to dryness under reduced pressure to provide (3-benzyl-2-oxo-2,3-dihydrobenzoimidazol-1-yl)-acetic acid (97% yield). Mp 200-202° C.; ¹H NMR (400 MHz, DMSO-d₆) δ ppm 4.65 (s, 2 H), 5.07 (s, 2 H), 6.97-7.07 (m, 2 H), 7.08-7.13 (m, 1 H), 7.15-7.19 (m, 1 H), 7.21-7.41 (m, 5 H), 13.09 (s, 1 H); ¹³C NMR (DMSO-d₆) δ 169.58, 153.77, 136.91, 129.28, 128.76, 128.60, 127.48, 127.32, 121.19, 108.30, 43.71, 42.07. COSY-NMR experiments show that C-4/C-7 and C-5/C-6 have the same δ; MS ESI: m/z (%) 283 (M+H⁺, 100); Anal. calc. for C₁₆H₁₄N₂O₃: C-68.08; H-5.00; N-9.92. Found: C-67.80; H-4.93; N-9.93.

(D) To a solution of t-butyl methyl malonate (1.91 g, 11 mmol) in DMF (10 mL) was added NaH (0.849 g, 21 mmol, 60% dispersion in oil) at 0° C. The reaction mixture was stirred for 1 h at 0° C., after which 4-fluoro-nitrobenzene (1.41 g, 10 mmol) was added. The reaction mixture was stirred for 12 h at RT, then poured into water (100 mL) and the product extracted with EtOAc (100 mL). The organic layer was washed with water (3×100 mL), dried over Na₂SO₄, and evaporated under vacuum. The crude product was purified by column chromatography (SiO₂, hexanes:EtOAc 9:1) to provide 2-(4-nitrophenyl)-malonic acid t-butyl ester methyl ester (2 g, 68% yield).

Proceeding as in part (A) above, but substituting 2-(4-nitrophenyl)-malonic acid t-butyl ester methyl ester for the starting material, and heating for 4 h at 100° C., the product (4-nitro-phenyl)-acetic acid methyl ester was produced (82% yield). Mp 50-52° C. (lit. 52-53° C.); ¹H NMR (400 MHz, CDCl₃) δ ppm 3.72 (s, 3 H), 3.75 (s, 2 H), 7.46 (d, J=8.08 Hz, 2 H), 8.19 (d, J=9.09 Hz, 2 H); ¹³C NMR (CDCl₃) δ ppm 170.56, 147.15, 141.21, 130.26, 123.70, 52.34, 40.72; MS ESI: m/z (%) 194 (M−H⁻, 100); Anal. calc. for C₉H₉NO₄: C-53.39; H-4.65; N-7.18. Found: C-55.41; H-4.62; N-7.40.

(E) Similarly, proceeding as in part (A) above, but substituting cyano-(4-nitrophenyl)-acetic acid t-butyl ester for the starting material, and heating at 100° C. for 1 h, the product (4-nitrophenyl)-acetonitrile was produced in 96% yield. Mp 113-115° C. (lit. 115-117° C.); ¹H NMR (400 MHz, CDCl₃) δ ppm 3.90 (s, 2 H), 7.55 (d, J=8.59 Hz, 2 H), 8.26 (d, J=8.59 Hz, 2 H); ¹³C NMR (CDCl₃) δ ppm 147.77, 136.99, 128.93, 124.31, 116.42, 23.55; Anal. calc. for C₈H₆NO₂: C-59.26; H-3.73; N-17.28. Found: C-58.89; H-3.69; N-16.96.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective spirit and scope of the present invention. All such modifications are intended to be within the scope of the claims appended hereto.

What is claimed:

1. A method for deprotecting a protected compound having a t-butyl ester or BOC carbonate protecting group, said method comprising:
a) dissolving a protected compound having a t-butyl ester or BOC carbonate protecting group in a fluorinated alcohol to form a solution;
b) heating said solution for a period of time sufficient to remove t-butyl or BOC from said protected compound, thereby providing a deprotected compound.

2. The method of claim 1, wherein said heating comprises heating by microwave radiation.

3. The method of claim 1, further comprising:
c) recovering said deprotected compound from said solution.

4. The method of claim 1, wherein said fluorinated alcohol is selected from the group consisting of 2,2,2-trifluoroethanol and 1,1,1,3,3,3-hexafluoroisopropanol.

5. The method of claim 4, wherein said fluorinated alcohol is 2,2,2-trifluoroethanol.

6. The method of claim 4, wherein said fluorinated alcohol is 1,1,1,3,3,3-hexafluoroisopropanol.
